(19) United States
(12) Patent Application Publication
(10) Pub. No.: US 2013/0260365 A1
(43) Pub. Date: Oct. 3, 2013

(54) HIGHLY EFFICIENT PROCESS FOR PRODUCING BIOETHANOL

(71) Applicant: HONDA MOTOR CO., LTD., Tokyo (JP)

(72) Inventors: Kosuke Murata, Saitama (JP); Yoshiki Tsuchida, Saitama (JP); Naoki Ohta, Saitama (JP)

(73) Assignee: HONDA MOTOR CO., LTD., Tokyo (JP)

(21) Appl. No.: 13/791,273

(22) Filed: Mar. 8, 2013

(30) Foreign Application Priority Data
Mar. 30, 2012 (JP) 2012-081746
Aug. 31, 2012 (JP) 2012-192564

(51) Int. Cl. C12Q 3/00

(52) U.S. Cl. CPC C12Q 3/00
USPC 435/3

(57) ABSTRACT

In SSF, a highly efficient process for producing bioethanol is provided. Concentrations of an organic acid formed in a saccharified solution are measured from the initiation to completion of the saccharification, an average change rate of the organic acid concentrations is determined based on the organic acid concentration levels at respective time points, and the optimal timing of feeding a fermentative microorganism cell to the saccharified solution is determined using the average change rate as the indicator, whereby the fermentative microorganism cell is fed.

### BACKGROUND OF THE INVENTION

1. Field of the Invention

The present invention relates to a process for producing bioethanol highly efficiently in the Simultaneous Saccharification and Fermentation (hereinafter referred to as “SSF”) wherein, for the production of alcohol from a biomass, the saccharification and fermentation of a pretreated biomass are carried out simultaneously. SSF is a technique in which the enzymatic saccharification of hemicellulose and cellulose takes place together with the ethanol fermentation of the saccharified product by fermentative microorganism cells in the same tank, and is a predominant means to simplify the process of alcohol production.

The present invention, in particular, relates to the improvement of fermentation efficiency by determining the time at which a fermentation inhibitory substance is produced during SSF and a concentration thereof, based on which an enzyme and fermentative microorganism cells are added at the optimal timing.

2. Description of the Related Art

The production steps of ethanol from a lignocellulosic biomass using a fermentative microorganism cell is roughly divided into four steps of pretreatment, saccharification, fermentation and concentration.

Of these four steps, the saccharification and fermentation are carried out simultaneously in SSF. Saccharides used herein for the fermentation include hexose and pentose. In SSF, the enzymatic saccharification and fermentation by a fermentative microorganism cells are carried out in the same chamber, or the saccharification and fermentation are carried out using a new microorganism cell imparted with both characteristics or conjugated microorganism cells. For this reason, SSF is a predominant means to simplify the process and expected to enhance the fermentation efficiency.

However, SSF is the technique which has been traditionally used for brewing Japanese rice wine and thus many inventions have limited purposes, e.g., specialization of target materials such as alcoholic beverages, leaving many of the optimal conditions for SSF using a biomass unknown. As a result, in the production process of ethanol from a biomass, the optimization of conditions to carry out the reaction highly efficiently is demanded.

Patent Literature 1 discloses a process wherein, in SSF using cellulose as a raw material, ethanol is efficiently obtained by decreasing a reaction temperature stepwise or continuously from the initial saccharification temperature during the simultaneous saccharification reaction.

However, Patent Literature 1 uses SSF to avoid the competitive inhibition and lacks in specifying an inhibitory substance contained in a sugar solution derived from a biomass and considering how to treat it.

Patent Literature 2 describes a process wherein the pH of enzyme reaction is preset at a higher value than the optimal pH at the adjustment stage before SSF is carried out using a cellulosic raw material such as paper. The pH is already known to decrease as SSF proceeds and thus the pH value is preset higher in consideration of the value expected to decrease.

However, the process described in Patent Literature 2 reduces the saccharification efficiency in SSF due to the pH set higher than the optimal value for the enzyme activity. Additionally the higher pH further is likely to cause sundry microorganism cell growth and contamination increase, thereby reducing the fermentation efficiency.

### CITATION LIST

### Patent Literature

Patent Literature 1: Japanese Patent Laid-Open No. 2010-246422
Patent Literature 2: Japanese Patent Laid-Open No. 2011-055715

### SUMMARY OF THE INVENTION

The factors which have been considered to affect the microorganic growth include the sugar concentration, pH and organic acid concentration in a sugar solution containing slurry condition derived from a biomass. An object of the present invention is to efficiently carry out the fermentation by analyzing the behaviors during SSF of these factors which affect microorganism cells while controlling the inhibitory factors of fermentative microorganism growth.

An object of the present invention, in production of alcohol from a biomass, is to solve the problem of reduction in fermentation efficiency caused by the stasis or death of the fermentative microorganism cells due to inhibitory substances produced during the saccharification and fermentation and to carry out the fermentation in the environment with little growth inhibition and fermentation inhibition.

The present invention solves the above problems found in SSF and provides a production process wherein an organic acid, an inhibitory substance, is detected with high sensitivity, the time at which the organic acid is formed is determined and a fermentative microorganism cell is fed before the organic acid, an inhibitory substance, is formed, thereby enhancing the fermentation efficiency in SSF.

The present inventors analyzed the behaviors of fermentation in SSF and found that organic acids such as acetic acid and formic acid, act as a fermentation inhibitory substance, whereby the present invention was conceived. Focusing on the timing at which an organic acid is formed during SSF and further a relationship between the dissociation constant of the formed organic acid and pH, conditions for the efficient formation of ethanol are determined based on these behaviors.

The process for producing alcohol of the present invention comprises a pretreatment step of obtaining a pretreated material for saccharification from a lignocellulosic biomass as a substrate, a saccharification step of obtaining a saccharified solution by saccharifying the pretreated material for saccharification using a saccharogenic amylase and a fermentation step of obtaining a fermented solution containing alcohol by fennenting the saccharified solution in the same chamber using a fermentative microorganism cell, the process further comprising a measurement step of determining concentration levels of an organic acid formed in the saccharified solution by a measuring unit from the initiation to completion of the saccharification and a fermentative microorganism cell feeding step of feeding the fermentative microorganism cell by predetermining an average change rate of the organic acid concentration per unit of time based on the organic acid concentration levels determined at respective time points by the measuring unit, and by determining a timing of feeding the fermentative microorganism cell before the average change rate increases in consideration of a time length required for the fermentative microorganism cell to grow.

It has been considered that the pH decrease, which is caused by various organic acids and carbon dioxide gas formed by the reaction of a saccharogenic amylase and activity of a fermentative microorganism cell, inhibits the growth of fermentative microorganism cell. However, the present inventors found that acetic acid, or the like, i.e., specific organic acids rather than the pH decrease caused by the above factors inhibit the growth of yeast, whereby the present invention was accomplished.

The present inventors analyzed the time course of organic acid formation in the saccharification reaction and found that the organic acid concentration abruptly elevates at a certain time point. Depending on the reaction conditions such as the enzyme used and temperatures, when rice straw, for example, is used as the biomass and Acremonium cellulase is used as the saccharogenic amylase, the organic acid concentration maintains a constant concentration for about 100 hours from the start of saccharification reaction without substantial increase. Then, the organic acid concentration abruptly increases at about 100 hours from the start of saccharification. When an average change rate per unit of time of the organic acid is calculated, the rate varies significantly at about 100 hours when the organic acid concentration abruptly increases, the time point at which the organic acid increases can be thus detected with high sensitivity using the average change rate as an indicator.

According to the timing of feeding a fermentative microorganism cell of the present invention, a fermentative microorganism cell may be fed at any time after the saccharification step is initiated insofar as the growth of fermentative microorganism cell can reach the stationary phase before the organic acid abruptly increases. Thus, when the timing of feeding a fermentative microorganism cell is set in this way, the fermentative microorganism cell used can grow sufficiently before the organic acid, which causes the fermentation inhibition, abruptly increases, owing to which the fermentative microorganism cell does not have to be under microorganism stasis or killed by the organic acid.

In the fermentative microorganism cell feeding step of the present invention, it is preferable that the fermentative microorganism cell be fed after the saccharification step is initiated and then a sugar concentration reaches a level at which the fermentative microorganism cell can grow but before the average change rate of the organic acid concentration increases, in consideration of the time length required for the fermentative microorganism cell to grow.

Immediately after the treatment is initiated using a saccharogenic enzyme, a sugar content is low and the growth of fermentative microorganism cell fed is hence limited. For this reason, the timing of feeding the fermentative microorganism cell after a sugar concentration is increased to a certain extent by a saccharogenic enzyme so as not to suppress the growth of fermentative microorganism cell results in better efficiency. This is because the growth of fermentative microorganism cell is better when a sugar concentration is higher, thereby efficiently producing alcohol. However, the fermentative microorganic count at the time of feeding is extremely lower than the fermentative microorganic count after the fermentative microorganism cell increases and reaches the stationary phase, and thus the sugar concentration required by the fermentative microorganism cell may also be low. Accordingly, the timing for feeding does not have to wait for a sugar concentration to reach the peak value but may be any time after a sugar concentration reaches a level which the fermentative microorganism cell to be fed requires for growth.

The step of feeding the fermentative microorganism cell of the present invention is carried out at the time at which the ratio of average change rates of the organic acid concentration reaches 20, minus the time length required for the fermentative microorganism cell to grow.

It was revealed that the ratios of average change rates taken before and after the organic acid concentration abruptly increases range 20 to 3200 times, depending on the type of organic acid and reaction temperature conditions. When the ratio of average change rates per unit of time is used as the indicator, the organic acid concentration remarkably increases at the time point or after the ratio exceeds 20 times, thereby causing the growth inhibition of fermentative microorganism cell. Thus, using the ratio of average change rates as the indicator, the fermentative microorganism cell may be allowed to sufficiently grow before the organic acid concentration increases.

The time length required for the growth varies depending on the kind of fermentative microorganism cell to be used, but the timing of feeding a fermentative microorganism cell may be determined in consideration of the time length required for the fermentative microorganism cell to grow so that the fermentative microorganism cell can grow sufficiently before the organic acid abruptly increases.

The present invention further comprises determining the organic acid concentration level as a product of an undissociation degree of the organic acid determined at a pH of the saccharified solution and a concentration of all organic acids actually measured for the saccharified solution.

The organic acid is in the equilibrium state between the dissociated form in which the hydrogen ion is released from an acid in a solution and the undissociated form in which the hydrogen ion is not released. Such an equilibrium state depends on the pH.

The organic acid, in the state of undissociated form, is known to easily permeate the cell membrane of a microorganism. For this reason, the step for feeding the fermentative microorganism cell needs to be determined based on the undissociated organic acid concentration. However, it is not common to directly determine an undissociated organic acid concentration in a solution.

Each organic acid has a specific dissociation constant and the presence ratio of a dissociated form to an undissociated form is hence determined by the pH of a solution. Accordingly, given all organic acid concentrations and the pH, an undissociated organic acid concentration can be determined by calculation. Thus, the undissociated organic acid concentration may be calculated from all organic acid concentrations in a solution and the pH thereof.

The concentration of undissociated organic acids in the actual saccharified solution is determined by determining all organic acid concentrations after all organic acids are caused to be undissociated form in the acid region and determining a concentration of undissociated organic acids at the pH of the actual saccharified solution based on the undissociation degree of the organic acids determined based on the pH.

The present invention further comprises using acetic acid, formic acid, lactic acid, succinic acid, tartaric acid, citric acid or maleic acid to obtain an organic acid concentration level for determining an average change rate.

The present inventors revealed that, as an organic acid, acetic acid, formic acid, lactic acid, succinic acid, tartaric acid, citric acid or maleic acid abruptly increases at the same timing and the timing of feeding the fermentative microorganism cell can be determined from the average change rate when any of these acids is used as an indicator. Consequently, an efficient alcohol production becomes viable by selecting and measuring an organic acid with good measurement accuracy in accordance with the system of SSF such as biomass and saccharogenic enzyme to be used.

The fermentative microorganism cell feeding step of feeding the fermentative microorganism cell of the present invention is carried out 5 to 50 hours before the time point at which the average change rate increases.

This is because the sufficient microorganic growth requires 5 to 50 hours depending on the kind of fermentative microorganism cell to be used, temperature conditions to carry out SSF, and the like.

### BRIEF DESCRIPTION OF THE DRAWINGS

FIG. 1 is a chart showing the growth curve of a fermentative microorganism cell in sugar solutions derived from rice straw biomass;

FIG. 2 is a chart showing organic acid concentrations contained in a sugar solution derived from rice straw biomass;

FIG. 3 is a chart showing the growth inhibition of fermentative microorganism cell caused by organic acids;

FIG. 4A is a chart showing the changes in acetic acid concentration accompanying the enzymatic saccharification reaction, and FIG. 4B is a chart showing the changes in glucose level;

FIG. 5 is an image illustrating the optimal timing of feeding a fermentative microorganism cell for highly efficient production of bioethanol; and

FIG. 6 is a chart showing the results of a model experiment which was carried out under the conditions of feeding a fermentative microorganism cell at the optimal timing of feeding the fermentative microorganism cell and feeding the microorganism cell after an organic acid increased.

### DETAILED DESCRIPTION OF THE PREFERRED EMBODIMENTS

The factors which have been considered to affect the microorganic growth include the sugar concentration, pH and organic acid concentration in a sugar solution containing slurry condition derived from a biomass. The present inventors revealed that the factor most affects the microorganic growth is organic acids among these factors. Accordingly, the present inventors determined the timing of feeding a fermentative microorganism cell using the increase of these organic acids as the indicator and enabled ethanol to be formed efficiently.

The present invention is described in detail with reference to the drawings.

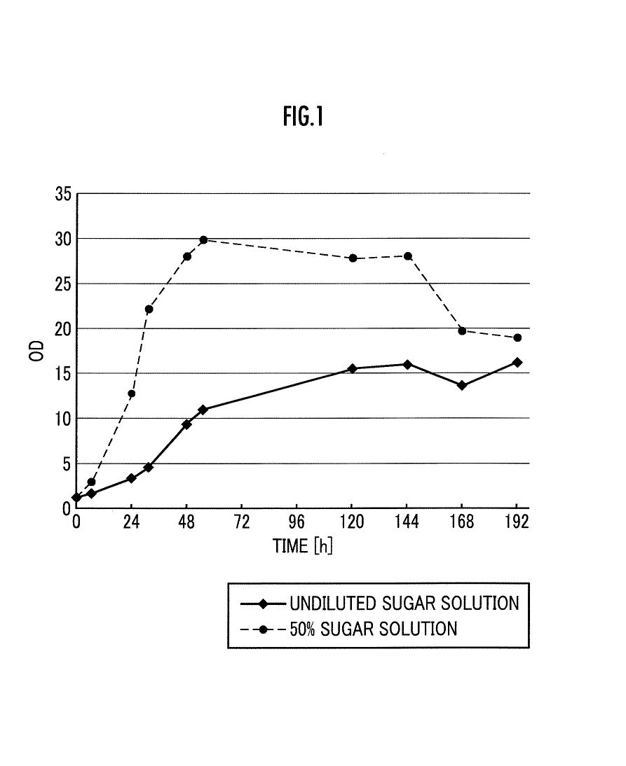

A fermentative microorganism cell (yeast) was added to a sugar solution derived from rice straw which was pretreated with ammonia and enzymatically saccharified and to a 50% sugar solution obtained by diluting the sugar solution, and the microorganic growths were measured using an absorption spectrometer. When an inhibitory substance is present, the dilution of the inhibitory substance is supposed to enable the fermentative microorganism cell to grow (FIG. 1).

As a result, the 50% sugar solution obtained by diluting an undiluted solution to 50% was found to have more microorganic growth than the undiluted sugar solution containing slurry condition of rice straw (FIG. 1). The fermentative microorganism cell grew better in the 50% sugar solution than in the undiluted sugar solution, based on which a substance which inhibits the fermentation is presumably contained in the sugar solution. The microorganic growth inhibition was also found, in addition to rice straw, when other lignocellulosic biomasses such as bagasse or corn stover were used.

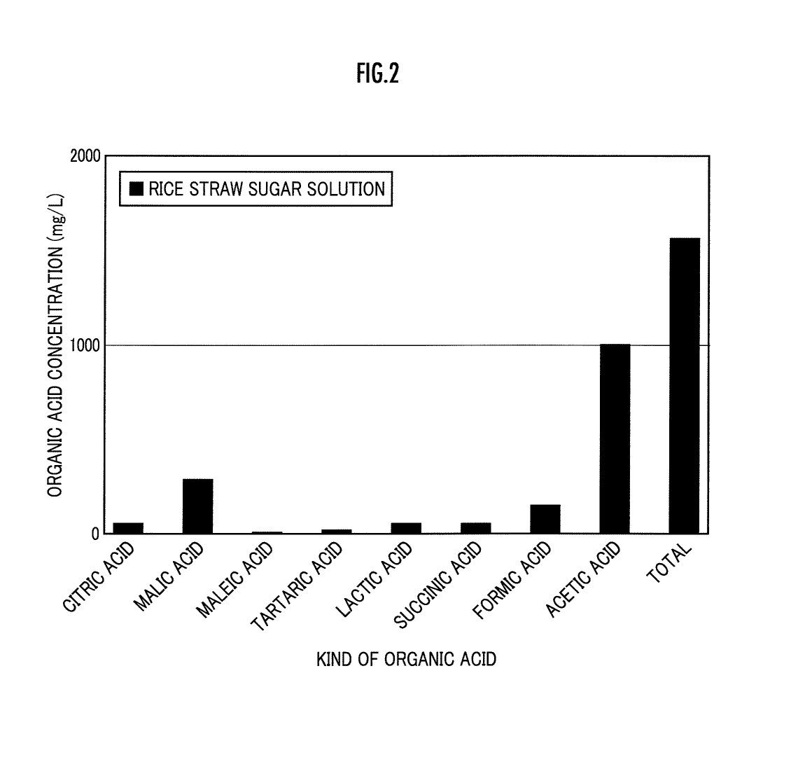

As described above, when an organic acid concentration exceeds the critical value, the activity of fermentative microorganism cell is suppressed by the microorganism stasis effect of the organic acids. This is one of the factors which cause a reduced fermentation efficiency. The mechanism of microorganism stasis caused by organic acids is considered to lie in the microorganic pH which is lowered by the proton released from the organic acids taken up by the microorganism cell. The organic acids, in the state of undissociated form, easily permeate the cell membrane of a microorganism cell and exhibit the microorganic stasis effect when the pH in the microorganism cell is lowered. Under the circumstances, the organic acids contained in a sugar solution derived from rice straw and the concentrations thereof were analyzed (FIG. 2).

In the light of the microorganic stasis mechanism of the organic acids described above, the undissociated organic acid concentration in each sugar solution needs to be measured. However, it is not common to directly determine the undissociated organic acid concentration in each sugar solution. In reality, the saccharified solutions are adjusted to different pH regions because the optimal pH region varies depending on the kind of saccharogenic amylase. The pH also changes in a time-dependent manner during the saccharification.

For these reasons, whole organic acid concentrations including both dissociated and undissociated acid are measured to calculate the undissociated organic acid concentration based on the pH of solution and the dissociation constants. Each organic acid has a specific dissociation constant (pKa) and the presence ratio of a dissociated form to an undissociated form is hence determined by the pH of a solution.

The undissociated organic acid concentration in a saccharified solution can be determined, as shown in the following formula 1, as a product of a pH-dependent undissociation degree and a concentration of the undissociated form of all organic acids actually measured.

An undissociated organic acid concentration in a saccharified solution=(undissociation degree)×(whole organic acid concentrations)=((1/(1+10^{pH-pKa}))×(all organic acid concentrations) [Expression 1]

Using this method, whole organic acid concentrations were determined as actual measured values by HPLC and the undissociated organic acid concentration in a sugar solution derived from rice straw was determined (FIG. 2). Although not shown, the undissociated organic acid concentrations in sugar solutions derived from corn stover and bagasse were also analyzed at the same time. When an organic acid concentration is measured using HPLC, dilute sulfuric acid is used as the mobile phase and thus the organic acids are all caused to be in the undissociated form.

The organic acids herein are measured using HPLC but any other methods capable of measuring the kind of organic acid and concentrations thereof such as gas chromatography or capillary electrophoresis may be used.

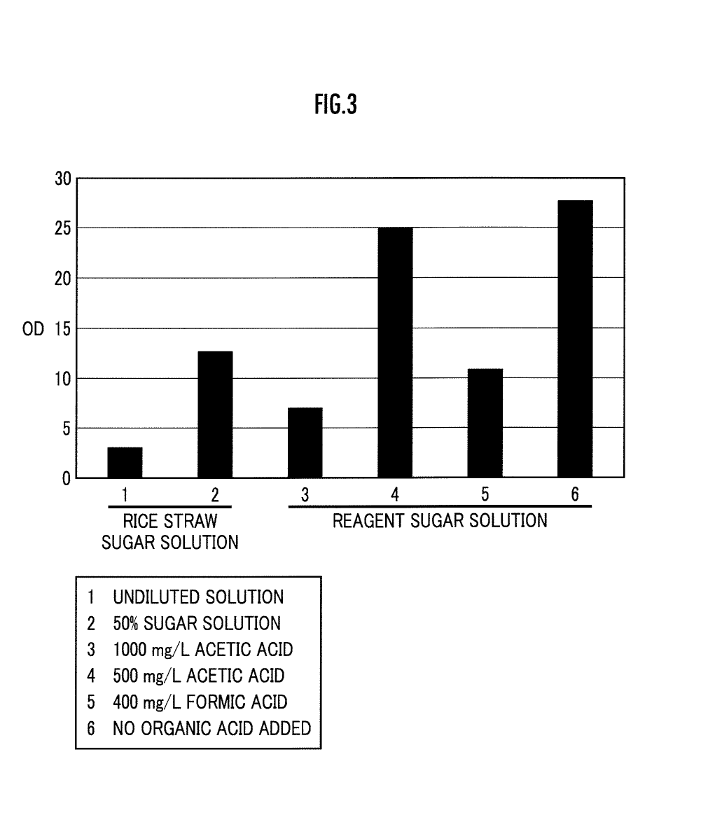

In addition to the sugar solution derived from rice straw shown in FIG. 2, various organic acids such as acetic acid and formic acid to start with, succinic acid, lactic acid, malic acid, citric acid, and the like, were detected in sugar solutions derived from corn stover and bagasse. Then, acetic acid and formic acid, which are detected relatively in high concentrations in any sugar solutions, were examined for the effects on the growth inhibition of a fermentative microorganism cell (FIG. 3).

Of the organic acids detected in sugar solutions derived from these biomasses, acetic acid or formic acid was added to a reagent sugar solution to prepare a sample with an adjusted concentration and the effects on the growth state of a fermentative microorganism cell by the addition of acetic acid or formic acid were observed. FIG. 3 shows the growth states of the fermentative microorganism cell after 24 hours.

Of the sugar solutions derived from the biomasses used for measuring the organic acid concentrations, the sugar solution derived from bagasse had the highest concentration of produced acetic acid of 3000 mg/L, whereas the culture broth to which 3000 mg/L of acetic acid was added to the reagent sugar solution had no growth of the fermentative microorganism cell. Then, a culture broth to which 1000 mg/L of acetic acid, which is equivalent to the undiluted sugar solution of rice straw, was added to the reagent sugar solution and a broth to which 500 mg/L of acetic acid, equivalent to the sugar concentration of a 50% rice straw sugar solution, was added to the reagent sugar solution were used to be the reagent sugar solutions.

The formic acid concentrations were 400 mg/L in the sugar solutions derived from corn stover and bagasse, which was higher than the formic acid concentration in the rice straw sugar solution (200 mg/L). For this reason, the formic acid concentration of 400 mg/L was employed, and 400 mg/L of formic acid was added to the reagent sugar solution to analyze the growth of fermentative microorganism cell.

The growths in the undiluted sugar solution of rice straw and in the 50% sugar solution were also analyzed at the same time under the same culture conditions as shown in FIG. 1. The undiluted sugar solution of rice straw contains 1000 mg/L of acetic acid and 200 mg/L of formic acid, and the 50% sugar solution contains 500 mg/L of acetic acid, 100 ml/L of formic acid and further organic acids other than acetic acid and formic acid.

When acetic acid or formic acid was added to the reagent sugar solution (3 to 5 in FIG. 3), the growth inhibition of fermentative microorganism cell was observed in comparison with the case where no organic acid was added (6 in FIG. 3). When acetic acid was added to the reagent sugar solution, the growth inhibition was observed in an acetic acid concentration-dependent manner. In other words, the fermentative microorganism cell grew better in the broth to which acetic acid in a lower concentration of 500 mg/L was added to the reagent sugar solution (4 in FIG. 3) than the broth to which 1000 mg/L of acetic acid was added to the reagent sugar solution (3 in FIG. 3).

When the rice straw sugar solution was used, like the result shown in FIG. 1, the obtained results suggest that the 50% sugar solution shown as 2 in FIG. 3 had better growth of the fermentative microorganism cell than the undiluted sugar solution of rice straw shown as 1 in FIG. 3. The acetic acid concentration of 1000 mg/L is the concentration equivalent to the undiluted sugar solution of rice straw, but the fermentative microorganism cell grows better when cultured in the reagent sugar solution to which 1000 mg/L of acetic acid was added as shown as 3 in FIG. 3 than when cultured using the undiluted sugar solution of rice straw shown as 1 in FIG. 3. This is presumably because other organic acids such as formic acid are not contained in the reagent sugar solution.

Further, the growth inhibition of fermentative microorganism cell was confirmed to take place by acetic acid and formic acid in a concentration-dependent manner (Tables 1 and 2). Sugar solutions to which acetic acid or formic acid was added were prepared for comparison with a reagent sugar solution containing no organic acid, and the fermentative microorganism cell of the same count was added to each sugar solution and cultured. At this time, the pHs of the reagent sugar solutions were adjusted to be the same. The absorbance of the microorganic counts was measured 24 hours later. Using the sugar solution to which acetic acid or formic acid was not added as the criterion, the degrees of growth inhibition are shown below. As the acetic acid or formic acid concentration increases, the fermentation inhibition increases. Consequently, it was confirmed that acetic acid and formic acid are the growth inhibition factors of a fermentative microorganism cell.

TABLE 1

| Acetic acid concentration (mM) | Growth inhibition (%) |
|---|---|
| 0 | 0 |
| 5.9 | 10.4 |
| 11.9 | 28.8 |
| 17.8 | 75.3 |

TABLE 2

| Formic acid concentration (mM) | Growth inhibition (%) |
|---|---|
| 0 | 0 |
| 10.0 | 5.6 |
| 20.0 | 61.1 |
| 30.0 | 71.4 |

Thus, the fact that the growth inhibition of a fermentative microorganism cell was caused in an organic acid concentration-dependent manner in the reagent sugar solution, to which an organic acid was added while the pH and sugar concentration were constantly maintained, suggests the significant involvement of the organic acids with the growth inhibition of a fermentative microorganism cell.

Furthermore, a rice straw, corn stover or bagasse solution was actually measured for the pH and sugar concentration and it was confirmed that the pH was close to 4.5, which is within the optimal pH range for a saccharogenic amylase, and the sugar concentration was sufficient for a fermentative microorganism cell to grow.

As described above, the factor which affects the growth of a fermentative microorganism cell is the concentration of organic acids such as acetic acid and formic acid rather than the sugar concentration in and pH of the sugar solution. Then, the fermentation efficiency can be enhanced when the time at which the organic acid increase is analyzed to adjust the timing of feeding a fermentative microorganism cell and the fermentative microorganism cell is fed before the inhibitory substances such as acetic acid increase and allowed to sufficiently grow.

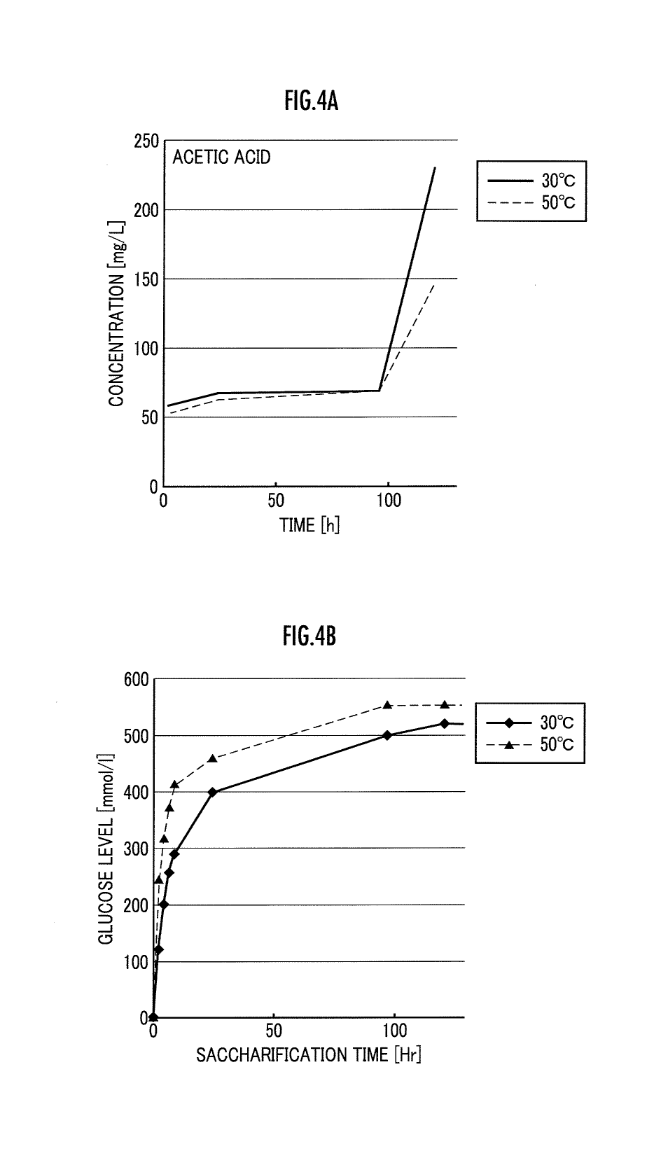

Accordingly, after feeding a saccharogenic amylase to a sugar solution obtained by pretreating a biomass, the time-course changes in concentration of acetic acid, which is present in a high concentration in sugar solutions among other organic acids, correlated with the growth inhibition of fermentative microorganism cell, were analyzed (FIG. 4A).

The saccharification was carried out by pretreating rice straw used as a biomass with 25 to 30% aqueous ammonia and using Acremonium cellulase (Meiji Seika Kaisha, LTD.).

Acremonium cellulase (Meiji Seika Kaisha, LTD.) was used herein as the saccharogenic amylase but other commercial saccharogenic amylases such as Ctec (Novozymes A/S) or Accellerase (Genencor Inc.) may be used.

As evident in FIG. 4A, the acetic acid concentrations substantially remained unchanged for up to about 100 hours at either temperature condition of 30° C. or 50° C. but abruptly elevate thereafter.

In FIG. 4A, the average change rates, i.e., the slopes of lines on the graph, are notably different before and after the acetic acid concentrations abruptly increase, based on which, when the acetic acid increase is detected using the average change rate as the indicator, the increase of acetic acid can be detected with high sensitivity. In this way the timing to initiate the fermentation can be determined before acetic acid, an inhibitory substance, increases.

The average change rate is determined by measuring organic acid concentrations (d1, d2) at different measuring times (t1, t2) and calculated as a value as linear function shown in the following formula 2.

Average change rate (slope)=(d2−d1)/(t2−t1) [Expression 2]

Although not shown, like the time-course change of acetic acid, formic acid substantially does not increase up to 100 hours after the addition of a saccharogenic amylase but abruptly increases at about 100 hours and the slope on the graph significantly changes. Consequently, the average change rate can be determined as the slope of two linear functions of before and after the organic acid abruptly increases.

TABLE 3
[Average change rates per unit of time of acetic acid and formic
acid at a reaction temperature of 30° C. and ratios]

| | Slope/before the | Slope/after the | Ratio of |
|---|---|---|---|
| | increase | increase | change rate |
| Acetic acid | 0.0701 | 6.76 | 96.4 |
| Formic acid | 0.0881 | 2.56 | 29.0 |

In Table 3, the ratio of slope before and after the organic acid abruptly increases is shown as the ratio of change rate (slope after the increase/slope before the increase).

As shown in Table 3, the ratio of average change rate can be used as the highly sensitive indicator for detecting the increase in acetic acid because it is 96.4 times in acetic acid and 29.0 times even in formic acid whose change is small.

Thus, using the increase in average change rates of an organic acid as the indicator, the time point at which the organic acid, an inhibitory substance, abruptly increases is predetermined and the timing of feeding a fermentative microorganism cell may be determined in expectation of the time length required for the fermentative microorganism cell to be fed to grow.

A fermentative microorganism cell may be fed at any time after the saccharification step is initiated insofar as the growth of fermentative microorganism cell can reach the stationary phase before the organic acid abruptly increases. In other words, a fermentative microorganism cell may be fed together with a saccharogenic amylase or may be fed after the saccharification proceeds and a sugar concentration reaches the steady state.

However, a fermentative microorganism cell favorably grows when a sugar concentration is above a certain level. Under the circumstances, the time-course changes of sugar concentration from the start of saccharification reaction by a saccharogenic amylase were subsequently analyzed to more accurately determine the timing of feeding a fermentative microorganism cell and glucose levels were measured.

The glucose level was measured using a biosensor. The glucose level was measured herein using a biosensor but may be analyzed using other methods such as HPLC.

A sugar concentration quickly increases after a saccharogenic amylase is fed to a pretreated biomass and the rate of increase decreases thereafter and proceeds to the gradual increase state where the sugar concentration slightly rises. When ammonia-treated rice straw is saccharified using Acremonium cellulase, the sugar concentration quickly starts elevating after the start of saccharification reaction and the rate of reaction slows down to the gradual increase state about 10 hours after the saccharification reaction started, whereby a saccharification rate reaches approximately 80% 24 hours later (FIG. 4B).

Thus, the sugar concentration has reached the sufficient level for a fermentative microorganism cell to grow after about 10 hours from the start of saccharification reaction.

The time length required for a fermentative microorganism cell to grow varies depending on the kind of fermentative microorganism cell but is about 5 to 50 hours. For this reason, the time at which organic acids such as acetic acid abruptly increases is determined based on the ratio of change rates and a fermentative microorganism cell may be fed, although varies depending on the strain, up to 5 to 50 hours before the determined time.

The above results suggest that the average change rate per unit of time is extremely useful as the indicator for determining the optimal timing of feeding a fermentative microorganism cell to a saccharified solution. Other organic acids were also analyzed for the time-course changes. As shown in Table 4, lactic acid, succinic acid, maleic acid, tartaric acid and citric acid were also found to abruptly increase after the saccharification was initiated at the same timing as acetic acid and formic acid. Consequently, these organic acids can also be used as the indicator for determining the timing of feeding a fermentative microorganism cell.

TABLE 4
[Average change rates per unit of time of lactic acid and succinic
acid at a reaction temperature of 30° C. and ratios]

| | Slope/before the | Slope/after the | |
|---|---|---|---|
| | increase | increase | Change rate |
| Lactic acid | 0.0304 | 2.61 | 85.8 |
| Succinic acid | 0.00210 | 6.72 | 3200.0 |
| Maleic acid | 0.000100 | 0.0398 | 398.0 |
| Tartaric acid | 0.0416 | 1.21 | 29.1 |
| Citric acid | 0.00790 | 0.769 | 97.4 |

Further, the average change rates of organic acids under a temperature condition of 50° C. were also determined because SSF is sometimes carried out at a temperature condition of 50° C. (Table 5).

TABLE 5
[Average change rates per unit of time of acetic acid, lactic acid and
succinic acid at a reaction temperature of 50° C. and ratios]

| | Slope/before the | Slope/after the | |
|---|---|---|---|
| | increase | increase | Change rate |
| Acetic acid | 0.0474 | 1.76 | 37.0 |
| Lactic acid | 0.0380 | 2.11 | 55.6 |
| Succinic acid | 0.145 | 3.04 | 20.9 |

These results show that even when a change rate is the lowest (succinic acid at a temperature condition of 50° C.), the ratio of average change rates is 20 or more due to which, according to the present invention, the elevation of organic acids, the growth inhibitory substances of a fermentative microorganism cell, can be detected with extremely high sensitivity.

All organic acids analyzed showed abrupt increases after the saccharification was initiated at the same timing as acetic acid and formic acid. It is thus conceived that any organic acids known to be associated with the saccharification can be used as the indicator for determining the timing of feeding a fermentative microorganism cell.

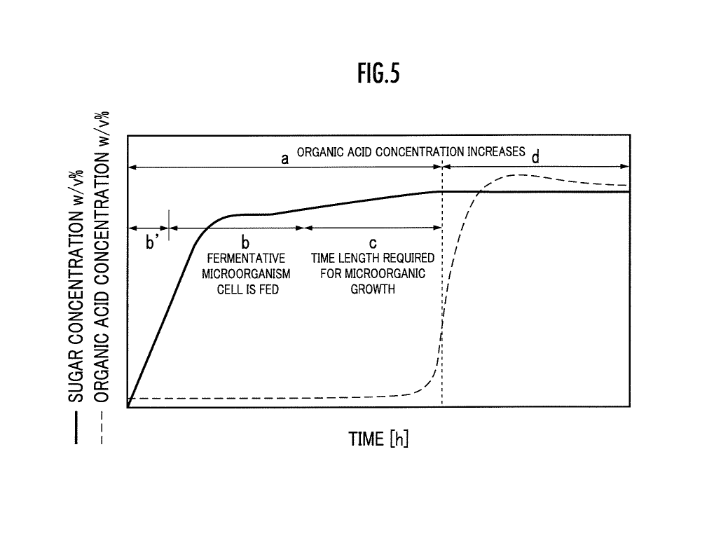

According to the above results, efficient production of ethanol is viable when a fermentative microorganism cell is fed before an organic acid concentration abruptly increases. In reality the timing varies depending on the enzyme to be used and the strain of a fermentative microorganism cell, but more optimally when a fermentative microorganism cell is fed before or after a sugar concentration gradually increases and before a concentration of an organic acid such as acetic acid abruptly increases in expectation of the time length required for the fermentative microorganism cell to sufficiently grow, highly efficient production of ethanol can be achieved (FIG. 5). More specifically, the timing of feeding a fermentative microorganism cell may be determined by predetermining the time at which an organic acid concentration abruptly increases based on a ratio of average change rates of an organic acid per unit of time in consideration of the growth time of the fermentative microorganism cell (FIG. 5c, approximately 5 to 50 hours) so that the fermentative microorganism cell can sufficiently ferment before the predetermined time. The feeding can be carried out at any time insofar as a fermentative microorganism cell can sufficiently grow before an organic acid abruptly increases (b and b′ in FIG. 5). Optimally, when a fermentative microorganism cell is fed shortly before a sugar concentration starts to be stabilized as shown as b in FIG. 5, the fermentative microorganism cell grows well and the fermentation proceeds more efficiently.

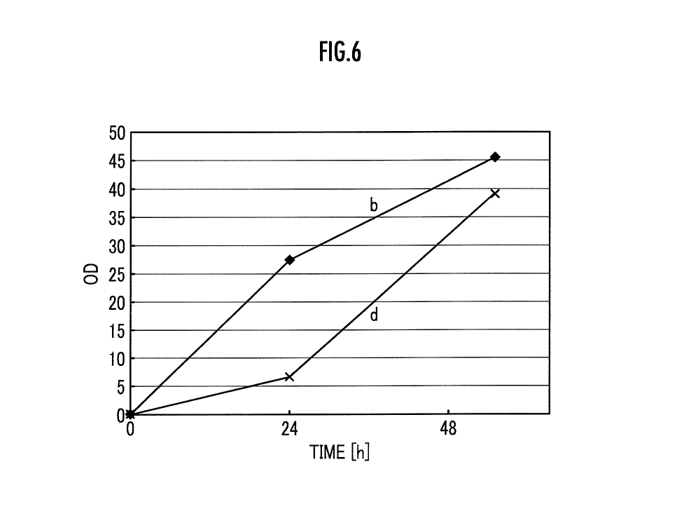

A model experiment was subsequently carried out. After 72 hours from feeding a microorganism cell to a rice straw sugar solution at 50° C. (a period corresponding to the period shown as d in FIG. 5), an acetic acid concentration was about 1000 mg/L. A sugar solution to which acetic acid was added so as to give 17.79 mM to be equivalent to this amount (equivalent to feeding a microorganism cell during the period shown as din FIG. 5) and a sugar solution to which acetic acid was not added (equivalent to a solution to which a fermentative microorganism cell is fed during the period shown as b in FIG. 5) were used to measure the microorganic growth by the absorbance (FIG. 6).

As shown in FIG. 6, the microorganic growth is evidently poorer in the solution (×) to which the microorganism cell was fed during the period corresponding to the period shown as d in FIG. 5 than in the solution (♦) to which the microorganism cell was fed during the period corresponding to the period shown as b in FIG. 5. Consequently, as shown in the present invention, when a time at which an organic acid abruptly increases is predetermined using an average change rate of an organic acid concentration as the indicator and a fermentative microorganism cell is fed considering the time length required for the fermentative microorganism cell to grow, alcohol is efficiently formed without inviting the growth inhibition of the fermentative microorganism cell.

According to this process, a fermentative microorganism cell is fed at a sufficient sugar concentration and before the concentration of organic acids, inhibitory substances, such as acetic acid and formic acid abruptly increases, thereby growing the microorganism cell without causing the growth inhibition thereof and fermenting efficiently.

Further, when the time point at which an organic acid increases is detected with high sensitivity and the timing of feeding a fermentative microorganism cell is determined according to the process of the present invention, the growth of fermentative microorganism cell is not inhibited and ethanol can be formed efficiently.

The sugar solution derived from rice straw was mainly used for the analysis of the above organic acid concentrations and the like, but without limiting thereto, other biomasses such as bagasse, corn stover, napier grass, switch glass, Miscanthus, Erianthus, sorghum, or Bermuda may also be used to determine the timing to add a fermentative microorganism cell, similarly using the ratio of average change rates of an organic acid concentration.

For the pretreatment, any treatment known as the biomass pretreatment such as hydrothermal treatment or supercritical pretreatment can be carried out in addition to the ammonia treatment.

According to the process of the present invention, in any SSF regardless the kind of biomass and pretreatment technique, the concentration change of an organic acid, a growth inhibitory substance of a fermentative microorganism cell, can be detected with high sensitivity and thus the timing of feeding a fermentative microorganism cell can be determined, thereby highly efficiently controlling the production of bioethanol.

What is claimed is:

1. A process for producing alcohol from a lignocellulosic biomass comprising:
a pretreatment step of obtaining a pretreated material for saccharification from a lignocellulosic biomass as a substrate;
a saccharification step of obtaining a saccharified solution by saccharifying the pretreated material for saccharification with a saccharogenic enzyme; and
a fermentation step of obtaining a fermented solution containing alcohol by fermenting the saccharified solution in the same chamber using a fermentative microorganism cell;
the process further comprising:
a measurement step of determining concentration levels of an organic acid formed in the saccharified solution by a measuring unit from the initiation to completion of the saccharification; and
a fermentative microorganism cell feeding step of feeding the fermentative microorganism cell by predetermining an average change rate of the organic acid concentration per unit of time from the organic acid concentration levels determined at respective time points by the measuring unit, and
by determining a timing of feeding the fermentative microorganism cell before the average change rate increases in consideration of a time length required for the fermentative microorganism cell to grow.

2. The process for producing alcohol from a lignocellulosic biomass according to claim 1,
wherein the fermentative microorganism cell feeding step further comprises a fermentative microorganism cell feeding step of feeding the fermentative microorganism cell after the saccharification step is initiated and then a sugar concentration reaches a level at which the fermentative microorganism cell can grow but before the average change rate increases, in consideration of the time length required for the fermentative microorganism cell to grow.

3. The process for producing alcohol from a lignocellulosic biomass according to claim 1,
wherein the fermentative microorganism cell feeding step is carried out by determining a ratio of the average change rates of the organic acid concentration per unit of time and timing the step by the time length required for the fermentative microorganism cell to grow earlier than a time at which the ratio reaches 20.

4. The process for producing alcohol from a lignocellulosic biomass according to claim 2,
wherein the organic acid concentration level is determined from a product of an undissociation degree of the organic acid determined at a pH of the saccharified solution and a concentration of an undissociated form of the organic acid actually measured for the saccharified solution.

5. The process for producing alcohol from a lignocellulosic biomass according to claim 1,
wherein the organic acid concentration level for determining the average change rate is obtained using acetic acid, formic acid, lactic acid, succinic acid, tartaric acid, citric acid or maleic acid.

6. The process for producing alcohol from a lignocellulosic biomass according to claim 1,
wherein the fermentative microorganism cell feeding step of feeding the fermentative microorganism cell is carried out 5 to 50 hours before a time point at which the average change rate increases.
